(19) United States
(12) Patent Application Publication
(10) Pub. No.: US 2018/0318105 A1
(43) Pub. Date: Nov. 8, 2018

(54) AN EXPANDABLE CAGE

(71) Applicant: TOBB EKONOMI VE TEKNOLOJI UNIVERSITESI, Cankaya/Ankara (TR)

(72) Inventors: Mesut Emre Yaman, Cankaya/Ankara (TR); Teyfik Demir, Cankaya/Ankara (TR); Tolga Tolunay, Yenimahalle/Ankara (TR)

(21) Appl. No.: 15/770,040

(22) PCT Filed: Oct. 24, 2016
(86) PCT No.: PCT/TR2016/000148

§ 371 Date: Apr. 20, 2018

(30) Foreign Application Priority Data
Oct. 26, 2015 (TR) 2015/13363

(51) Int. Cl. A61F 2/44

(52) U.S. Cl. CPC A61F 2/4455; A61F 2002/3052; A61F 2/442

(57) ABSTRACT

The invention relates to an expandable cage (1) which is placed between two vertebrae and expandable at a preferred ratio towards the vertebrae, and hence mountable in any intervertebral space.

### TECHNICAL FIELD

The present invention relates to an expandable cage which is placed between the vertebrae and capable of expanding in longitudinal axis of the vertebral column by means of a balloon.

### PRIOR ART

Discs made of articular fibrocartilage and allowing the vertebrae to move easily with respect to one another are present between human vertebrae. Two adjacent vertebrae can easily move relative to one another thanks to said discs, in six degrees of freedom. Discs are present between all successive vertebrae, thereby allowing a limited movement of the vertebral column. In case damage occurs in the vertebrae or vertebral column due to an accident, a tumor incidence, or old age, a part of the disc may be dislocated and apply pressure on the nerve roots and/or the spinal cord. In such cases, the so-called discectomy procedure is performed in which the whole, or a part of the disc is removed surgically. There remains a space between the vertebrae subsequent to discectomy. Said space results in a decrease in the paravertebral disc height. In this case, it may be desirable to join the related vertebral segments together by means of fusion. When fusion is to be performed, an interbody fusion device is positioned in said space such that it will maintain paravertebral height after discectomy. These devices are also referred to as vertebral cages.

Today, many of the cages used for filling the space between two vertebrae are not capable of expanding. In case of placing a cage that is not capable of expanding between the vertebrae, one of the cages having a standard height is placed by medium fit in accordance with a predetermined size, or the decision made during the surgery. This, in turn, makes it difficult to adjust paravertebral height. If the height of the cage is too much, two neighboring vertebrae may be spaced apart more than needed; or in the reverse situation, they may become loose in place. In the expandable systems, on the other hand, the expansion takes place over a side of the cage. The heights of the parallel surfaces of the cage contacting with the endplates change conically, but not in parallel, subsequent to expansion.

### OBJECTS OF THE INVENTION

The object of the present invention is to provide an expandable cage which, once expanded, does not reverse.

Another object of the invention is to provide an expandable cage the expansion ratio of which is controllable by means of a balloon.

### DETAILED DESCRIPTION OF THE INVENTION

The expandable cage provided in order to achieve the object of the invention is illustrated in the accompanying figures, in which;

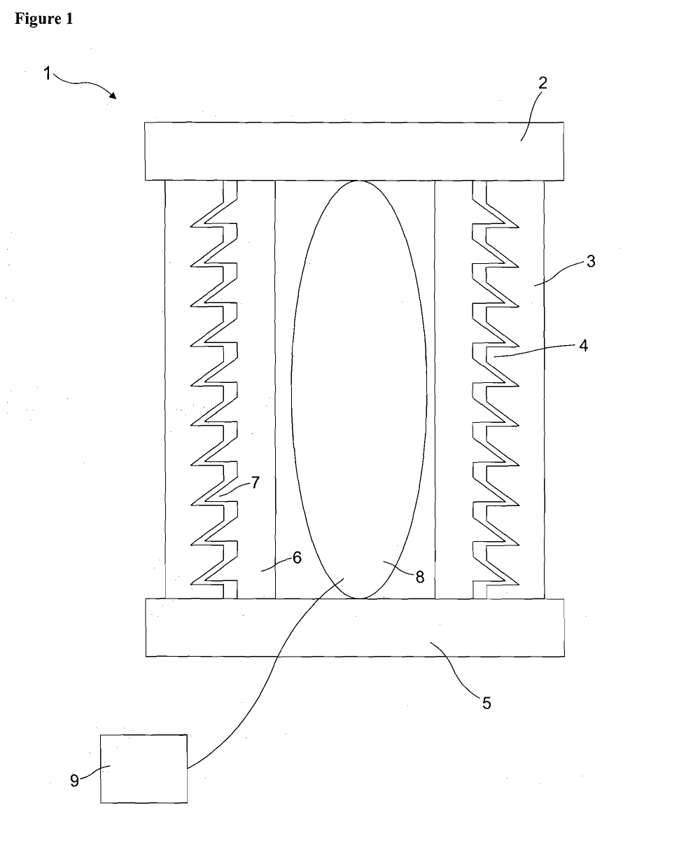

FIG. 1 is the schematic view of the expandable cage.

The parts in the figures are numbered individually and the references corresponding to the related part are listed below.

1. Expandable cage

2. Outer body

3. Outer extension

4. First nail

5. Inner body

6. Inner extension

7. Second nail

8. Balloon

9. Compression element

An expandable cage (1) which is placed between two vertebrae and expandable at a preferred ratio towards the vertebrae (i.e. in longitudinal axis), and hence mountable in any intervertebral space, and the height of which can be increased after being placed by being passed through a small opening, mainly comprises the following;
at least one outer body (2) which is expandable in longitudinal axis,
at least two outer extensions (3) disposed at the side of the outer body (2) which is not in contact with the vertebra,
at least one first nail (4) disposed at the side of the outer extensions (3) which is perpendicular to the outer body (2),
at least one inner body (5) which expands in the direction of the other vertebra which is not in contact with the outer body (2),
at least two inner extensions (6) disposed in the inner body (5) and extending in parallel to the outer extensions (3),
at least one second nail (7) disposed in the inner extension (6) and maintaining the distance between the outer body (2) and the inner body (5) by contacting with the first nail (4),
at least one balloon (8) which is positioned between the inner extensions (6) and moving the outer body (2) and the inner body (5) away from each other by applying pressure on the fluid therein, and
at least one compression element (9) allowing to apply pressure into the balloon (8).

The expandable cage (1) according to an embodiment of the invention is positioned between two vertebrae. The expandable cage (1) basically consists of an outer body (2) and an inner body (5) which is preferably slightly smaller than the outer body (2) and fits in said outer body (2). The outer body (2) is positioned such that it will contact with one of the vertebrae while the inner body (5) contacts with the other vertebrae which is not in contact with the outer body (2). The outer body (2) is provided with more than one outer extension (3) extending from the surface opposite the surface contacting the vertebra. This embodiment of the invention comprises two outer extensions (3). These outer extensions (3) are designed such that they will be shorter than the distance between the vertebrae. The first nail (4) is arranged at one side of the outer extension (3). The first nails (4) are continuous all along the outer extension (3) starting from the base of the outer body (2). The expandable cage (1) is provided with an inner body (5) as well as the outer body (2). The inner body (5) according to this embodiment of the invention is preferably slightly smaller than the outer body (2). Similar to the outer extensions (3) disposed in the outer body (2), the inner body (5) is provided with inner extensions (6). The second nail (7) is arranged at the side of the inner extensions (6) which faces the outer extension (3), said second nails (7) being in contact with the first nails (4). The first nail (4) and the second nail (7) disposed respectively in the outer extension (3) and the inner extension (6) allow moving only in one direction, while restraining movement in the other. The allowed direction to move by the first nail (4) and the second nail (7) is the direction in which the outer body (2) and the inner body (5) move away from each other. In cases when the outer body (2) and the inner body (5) move away from each other, said first nail (4) and second nail (7) slide over one another, and thus allowing the outer body (2) and the inner body (5) to move away from each other; on the other hand, in cases when the outer body (2) and the inner body (5) tend to get closer due to the pressure applied on the outer body (2) and the inner body (5), said first nail (4) and second nail (7) do not allow this movement. Moving the outer body (2) and the inner body (5) away from each other is ensured by means of a balloon (8) located inside the inner extension (6). Upon pumping a fluid into the balloon (8), said balloon (8) starts to inflate creating a volume. If this volume starts to get larger than the space between the outer body (2) and the inner body (5), then the balloon (8) applies pressure on the outer body (2) and the inner body (5), thereby moving the outer body (2) and the inner body (5) away from each other. Delivering a fluid into the balloon (8) is realized by way of a compression element (9). In this embodiment of the invention, the compression element (9) is a gun, said gun permitting injecting fluid into the balloon (8).

The expandable cage (1) according to this embodiment of the invention is used as follows. The ready-to-use expandable cage (1) which is of a suitable height to be inserted between two vertebrae is positioned between these vertebrae. Once the expandable cage (1) is positioned at the preferred site between the vertebrae, a fluid is compressed to balloon (8) disposed between the outer body (2) and the inner body (5) by means of the compression element (9). When said fluid is started to be injected into the balloon (8), the balloon (8) inflates, and after a while, it starts to apply pressure on the outer body (2) and the inner body (5). As a result of this pressure, the balloon (8) moves the outer body (2) and the inner body (5) away from each other. As a consequence of said distancing, the first nail (4) and the second nail (7) perform sliding movement on one another. Upon the contact of the outer body (2) with one vertebra, and that of the inner body (5) with the other, inflating the balloon (8) is stopped and the balloon (8) is removed from inside the inner body (5) and the outer body (2). Since the first nail (4) and the second nail (7) do not allow reverse movement once the balloon (8) is removed, the expandable cage (1) is positioned as it is, i.e. in its expanded form.

1. An expandable cage (1) which is placed between two vertebrae and expandable at a preferred ratio towards the vertebrae (i.e. in longitudinal axis), and hence mountable in any intervertebral space, and the height of which can be increased after being placed by being passed through a small opening, said expandable cage is characterized by mainly comprising;
at least one outer body (2) which is expandable in longitudinal axis,
at least two outer extensions (3) disposed at the side of the outer body (2) which is not in contact with the vertebra,
at least one first nail (4) disposed at the side of the outer extensions (3) which is perpendicular to the outer body (2),
at least one inner body (5) which expands in the direction of the other vertebra which is not in contact with the outer body (2),
at least two inner extensions (6) disposed in the inner body (5) and extending in parallel to the outer extensions (3),
at least one second nail (7) disposed in the inner extension (6) and maintaining the distance between the outer body (2) and the inner body (5) by contacting with the first nail (4), and
at least one balloon (8) which is positioned between the inner extensions (6) between the outer body (2) and the inner body (5), and into which a fluid is compressed by means of the compression element (9), and also which is moving the outer body (2) and the inner body (5) away from each other by applying pressure on the fluid therein.

2. The expandable cage (1) according to claim 1, characterized by at least one compression element (9) allowing to apply pressure into the balloon (8).

3. The expandable cage (1) according to claim 1, characterized in that the outer body (2) comprises an outer extension (3) extending from the surface opposite the surface contacting the vertebra.

4. The expandable cage (1) according to claim 1, characterized by the outer extensions (3) designed such that they will be shorter than the distance between the vertebrae.

5. The expandable cage (1) according to claim 1, characterized in that the outer extension (3) comprises a first nail (4) which is disposed at one side thereof and continuous all along the outer extension (3) starting from the base of the outer body (2).

6. The expandable cage (1) according to claim 1, characterized by an inner body (5) which is slightly smaller than the outer body (2) and has inner extensions (6).

7. The expandable cage (1) according to claim 1, characterized by a second nail (7) which is disposed at the side of the inner extensions (6) which faces the outer extension (3), and in contact with the first nails (4).

8. The expandable cage (1) according to claim 1, characterized by a first nail (4) disposed in the outer extension (3) in a way to allow moving only in one direction, while restraining movement in the other.

9. The expandable cage (1) according to claim 1, characterized by a second nail (7) disposed in the inner extension (6) in a way to allow moving only in one direction, while restraining movement in the other.

10. The expandable cage (1) according to claim 1, characterized by a balloon (8) which is located inside the inner extension (6) and moves the outer body (2) and the inner body (5) away from each other.

11. The expandable cage (1) according to claim 1, characterized by a balloon (8) which starts to inflate when a fluid is pumped thereinto and creates a volume.

12. The expandable cage (1) according to claim 1, characterized by a compression element (9) which allows delivering a fluid into the balloon (8).

13. The expandable cage (1) according to claim 1, characterized by a compression element (9) which is a gun and allows injecting fluid into the balloon (8).

14. (canceled)

15. The expandable cage (1) according to claim 1, characterized by a balloon (8) which inflates when fluid is started to be injected thereinto and starts to apply pressure on the outer body (2) and the inner body (5) and after a while.

16. The expandable cage (1) according to claim 1, characterized by a balloon (8) which moves the outer body (2) and the inner body (5) away from each other as a result of the pressure.
